(19) United States
(12) Patent Application Publication
(10) Pub. No.: US 2020/0010799 A1
(43) Pub. Date: Jan. 9, 2020

(54) SCAFFOLD FOR IN VITRO MODELING AND TRANSPLANTATION THERAPY OF PHOTORECEPTORS

(71) Applicant: Wisconsin Alumni Research Foundation, Madison, WI (US)

(72) Inventors: Zhenqiang Ma, Middleton, WI (US); Juhwan Lee, Madison, WI (US); Yei Hwan Jung, Madison, WI (US); Michael Phillips, Stoughton, WI (US); David Gamm, Waunakee, WI (US); Shaoqin Gong, Middleton, WI (US); Inkyu Lee, Madison, WI (US)

(21) Appl. No.: 16/506,471

(22) Filed: Jul. 9, 2019

Related U.S. Application Data
(60) Provisional application No. 62/695,403, filed on Jul. 9, 2018.

(51) Int. Cl. C12N 5/0793

(52) U.S. Cl. CPC C12N 5/062; C12N 2535/00; C12N 2533/30

(57) ABSTRACT

Photoreceptor scaffolds that can be used for transplantation of organized photoreceptor tissue, with or without retinal pigment epithelial cells, which may improve grafted cell survival, integration, and functional visual rescue are disclosed herein. The scaffolds include a cell support layer having at least one cube-shaped reservoir fluidly connected to a plurality of through-holes and at least one cell in the at least one cube-shaped reservoir.

### CROSS-REFERENCE TO RELATED APPLICATION

This application claims priority to U.S. Provisional Application Ser. No. 62/695,403, filed Jul. 9, 2018, which is incorporated by reference in its entirety.

### STATEMENT OF GOVERNMENT SUPPORT

This invention was made with government support under EY021218 and EY023497 awarded by the National Institutes of Health. The government has certain rights in the invention.

### BACKGROUND OF THE DISCLOSURE

The present disclosure relates generally to scaffolds for use in transplantation of organized photoreceptor tissue, with or without retinal pigment epithelium (RPE), which may improve grafted cell survival, integration, and functional visual rescue. Further, these scaffolds can be used in in vitro developmental and disease studies, as well as for drug screening.

Photoreceptors are crucial for vision, and they capture and transduce photons into electrochemical signals to be processed by the retina and visual centers of the brain. Adjacent to the photoreceptors are the retinal pigment epithelium (RPE), supportive cells required for photoreceptor health and function. All blinding disorders of the outer retina involve dysfunction and eventual degeneration of the photoreceptors, either alone (as occurs in many forms of retinitis pigmentosa) or with involvement of the RPE (as found with the prevalent disorder, age-related macular degeneration (AMD)). Currently, these patients have limited to no treatment options. One broadly applicable treatment strategy would be to replace photoreceptors alone or in combination with RPE.

The only approved embryonic stem cell (“ESC”) clinical trials currently underway in humans involve the transplantation of RPE via a simple, disorganized bolus injection of cells, or delivery of RPE on planar scaffolds. This work has demonstrated the safety of this approach. However, when photoreceptors are irreversibly lost, transplanting RPE alone will not rescue vision in advanced disease. For this to occur, light sensing photoreceptors must also be replaced. To make this prospect more complex, photoreceptors are a highly polarized, specialized cell type with apical outer segments containing light sensing photo-pigments and basal axon terminals. While transplantation of polarized photoreceptors with or without RPE presents significant challenges, microfabrication technology offers potential solutions to these issues.

Particularly, it would be advantageous if a scaffold could be prepared to provide polarization of photoreceptors, with or without RPE, to mimic native retinal tissues. It would be further advantageous if these scaffolds could provide a means to transplant organized photoreceptor tissue, with or without RPE, which may improve grafted cell survival, integration, and functional visual rescue compared to simple bolus cellular injections.

### BRIEF DESCRIPTION

The present disclosure is generally related to photoreceptor scaffolds that can be used for transplantation of organized photoreceptor tissue, with or without RPE, which may improve grafted cell survival, integration, and functional visual rescue. Particularly, the photoreceptor scaffold is structured from a biocompatible film having cube-shaped reservoirs, each reservoir patterned with an array of through-holes. These scaffolds can be produced from biodegradable and non-biodegradable materials.

In one aspect, the present disclosure is directed to a scaffold comprising a cell support layer comprising at least one cube-shaped reservoir, wherein the at least one cube-shaped reservoir is fluidly connected to a plurality of through-holes.

In another aspect, the present disclosure is directed to a cell culture scaffold system comprising a scaffold comprising a cell support layer comprising at least one cube-shaped reservoir, wherein the at least one cube-shaped reservoir is fluidly connected to a plurality of through-holes, and at least one cell in the at least one cube-shaped reservoir.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts one exemplary scaffold of the present disclosure.

FIG. 2A and FIG. 2B depict single cube-shaped cell reservoir dimensions (FIG. 2A) and through-hole dimensions and spacing (FIG. 2B) for use in the present disclosure.

FIG. 3 depicts a schematic illustration of one embodiment of the fabrication process for the silicon wafer to generate the hard-PDMS master mold for use in making the scaffold of the present disclosure.

FIG. 4A depicts one exemplary etched silicon wafer used in the step of silicon DRIE of the fabrication process as shown in FIG. 3.

FIG. 4B depicts a close-up view of the exemplary etched silicon wafer shown in FIG. 4A.

FIG. 4C depicts a cross-sectional view of the exemplary etched silicon wafer shown in FIG. 4A.

FIG. 5A depicts one exemplary hard-PDMS master mold generated from the etched silicon wafer fabricated in the fabrication process as shown in FIG. 3.

FIG. 5B depicts a close-up view of the exemplary hard-PDMS master mold shown in FIG. 5A.

FIG. 5C depicts a cross-sectional view of the exemplary hard-PDMS master mold shown in FIG. 5A.

FIG. 6A depicts one exemplary soft-PDMS scaffold used in the fabrication process as shown in FIG. 3.

FIG. 6B depicts a close-up view of the exemplary soft-PDMS scaffold shown in FIG. 6A.

FIG. 6C depicts a cross-sectional view of the exemplary soft-PDMS scaffold shown in FIG. 6A.

FIGS. 7A-7E depict a schematic illustration of a second embodiment of the fabrication process for a hard-PDMS master mold, and the micromolding process of the hard-PDMS master mold with desired scaffold material to fabricate an ice-cube tray photoreceptor scaffold.

FIGS. 8A-8C are photographic images depicting the fabrication process of PGS scaffolds as made using the process of FIGS. 7D-7E. FIG. 8A depicts a hard-PDMS master mold ready to be detached from the PGS scaffold after the curing process of PGS scaffolds. FIG. 8B depicts a PGS scaffold on a silicon wafer after the detachment of the hard-PDMS master mold. After removing the edge of scaffold, the scaffold is delaminated from the silicon wafer using a single edge razor blade. FIG. 8C depicts the fabricated PGS scaffold in the air.

FIGS. 8D-8F are SEM images of the fabricated ice-cube tray PGS photoreceptor scaffold showing (FIG. 8D) a top view and (FIG. 8E) a bottom view. FIG. 8F is a large area SEM image of the fabricated PGS scaffold.

FIGS. 9A-9F are photographic images showing the processes used to mount the photoreceptor scaffold on the transwell insert. FIG. 9A depicts a transwell insert with PGS scaffold below. The outer edge of the scaffold was attached to the transwell insert using a soft PDMS as a glue. The cell seeding area of the transwell insert was 78.5 mm² (internal diameter: 5 mm). FIG. 9B depicts a transwell insert holder grabbing a transwell insert. FIG. 9C depicts a 6-well cell co-culture system. FIGS. 9D-9F depict micro-patterned “ice-cube tray” scaffolds being readily filled with cells generated from a human pluripotent stem cell (hPSC)-derived CRX^{+/tdTomato} reporter line. Photoreceptors (PRs) are fluorescently labeled during differentiation and maturation of hPSC-derived retinal organoids, which were dissociated and seeded onto scaffolds for confocal imaging. FIG. 9D shows that 3D rendering (176 μm×185 μm×22 μm) confirmed successful capture of multiple CRX^{+/tdTomato}-expressing PRs (labeled in red) in individual wells of a laminin-coated scaffold. Cell nuclei are labeled with DAPI (blue). FIG. 9E depicts cells were seeded onto scaffolds at increasing concentrations (1, 3, 5, or 7 million/transwell) and imaged after 5 days in culture to determine the minimum concentration required to achieve the maximum number of CRX^{+/}tdTomato-PRs in each scaffold well. 5 million cells per transwell was ideal, as it was the minimum number of cells necessary to achieve maximal cell payload. FIG. 9F depicts that scaffolds seeded with CRX^{+/td/}Tomato-PRs (RFP+, red) contain both ARR3-expressing cone PRs (green) and NR2E3-expressing rod PRs (pink). A 3D side view of the scaffold demonstrates relatively even distribution of ARR3+ cones and NR2E3+ rods. 3D rendering was 644 μm×644 μm×20 μm.

FIGS. 10A-10D are finite element analysis showing equivalent von Mises stress distribution in the ice-cube tray structures with 5 N of tensile force applied on the PGS scaffold. The color bar shows the von Mises stress (in N/m²) for an applied tensile force. Isometric view (FIG. 10A), top view (FIG. 10B), bottom view (FIG. 10C), and side view (FIG. 10D).

FIGS. 11A-11D depict micro-patterned “ice-cube tray” scaffolds designed to act synergistically with existing host retinal structures, supporting prearranged orientation of seeded PRs. FIGS. 11A & 11C show that CRX^{+/}tdTomato-PRs (red) plated on scaffolds mature to express PRPH2 (green), a marker of photoreceptor (PR) outer segments, which are crucial structures that eventually contain photosensitive opsins in mature PRs. FIGS. 11B & 11D show that scaffolds also support organized expression of VGLUT1 (green), a presynaptic marker that primarily localizes to the top half of the scaffold.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The present disclosure is directed to a photoreceptor scaffold for photoreceptor delivery and polarization, with or without retinal pigment epithelium (RPE). The scaffolds can be used for in vitro developmental and disease studies, as well as for drug screening. These scaffolds can further be used for transplantation of organized photoreceptor tissue, with or without RPE, which may improve grafted cell survival, integration, and functional visual rescue.

Structure of Scaffold

Generally, a scaffold is provided for use in cell culture. The scaffold generally includes a cell support layer having at least one cube-shaped reservoir fluidly connected to a plurality of through-holes. The cell support layer is typically comprised of a biocompatible flexible polymer, and in some particularly suitable embodiments, the polymer is biodegradable. The polymer can be porous or non-porous. Suitable polymers include, but are not limited to, synthetic rubbers such as silicone rubbers (e.g., polydimethylsiloxane (PDMS)), polyurethane rubber, styrene butadiene rubber, and acrylonitrile butadiene rubber, natural rubbers (e.g., poly-cis-isoprene), thermoplastic elastomers (e.g., thermoplastic polyurethane, thermoplastic copolyester, thermoplastic polyamide), epoxies (e.g., SU-8), polyimides, polyurethanes, polyamides, polyesters (e.g., poly(lactic-co-glycolic acid) (PLGA), polylactic acid (PLA), polyglycolic acid (PGA), polycaprolactone (PCL), poly(glycerol sebacate) (PGS)), polysaccharides (e.g., chitosan), parylene, and combinations thereof. Advantageously, these polymers are “plastic-like” in that they are flexible upon application of an applied force, allowing for ease of transplantation and manipulation of the scaffold. In one particular aspect, the polymer is PDMS.

The cell support layer generally has at least one cube-shaped reservoir connected to the through-holes, the combination of which extends through the cell support layer. As used herein, “cube-shaped reservoir” refers to a cell reservoir having a cube shape (e.g., ice-cube tray design).

As used herein, “through-holes” refers to a channel that allows for guided growth of cells such as growth of the processes that will become the axons of a photoreceptor cell.

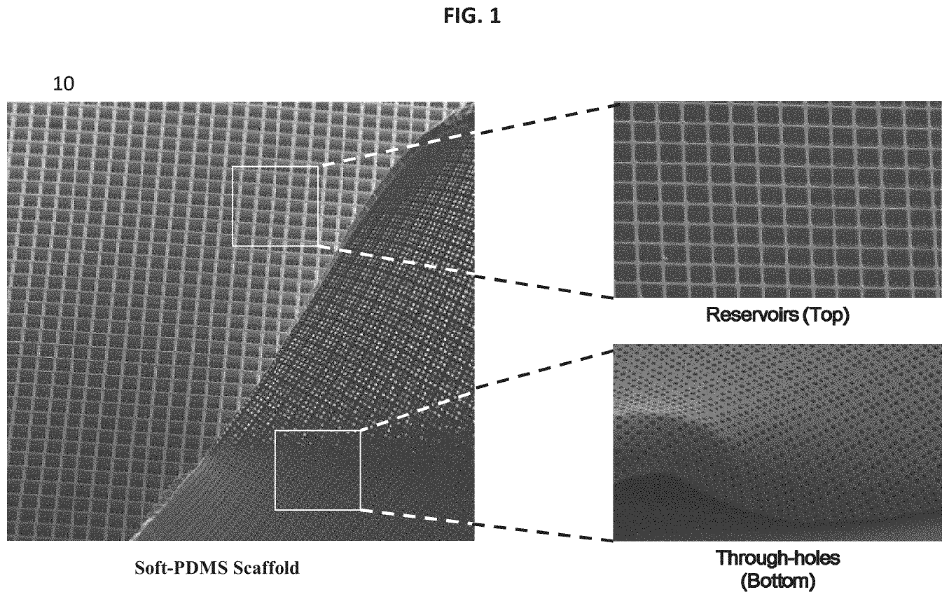

An exemplary cell support layer is shown in FIG. 1. As shown in FIG. 1, the cell support layer 10 includes at least one cube-shaped reservoir 12, each reservoir 12 fluidly connected to a plurality of through-holes 14. As used herein, a “plurality” refers to at least two through-holes, including at least 3, including at least 4, including at least 5, including at least 6, including at least 7, including at least 8, including at least 9, or more through-holes. While as shown, the cell support layer 10 includes a plurality of cube-shaped reservoirs 12, it should be understood by one skilled in the art that the cell support layer can include less than the plurality of cube-shaped reservoirs shown, including a single cube-shaped reservoir, or greater than the plurality of cube-shaped reservoirs shown without departing for the scope of the present disclosure.

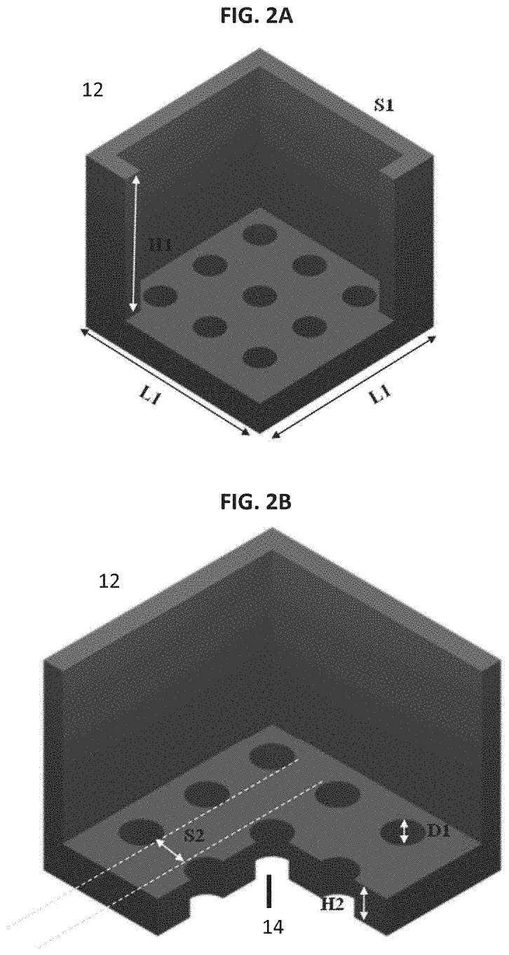

In one embodiment, as shown in FIG. 2A and FIG. 2B, the cube-shaped reservoir 12 includes a length, indicated at L1, that is larger than the diameter of the through-hole 14, indicated at D1 (FIG. 2B). In particularly suitable embodiments, the length of the cube-shaped reservoirs 12, L1, ranges from about 15 μm to about 100 μm. Further, a single cube-shaped reservoir has a height, H1, ranging from about 5 μm to about 100 μm.

The diameter of the through-hole 14, D1, typically ranges from about 1 μm to about 7 μm. Further, a single through-hole of the plurality of through-holes has a height, H2, ranging from about 1 μm to about 25 μm.

When a plurality of cube-shaped reservoir are fluidly connected to through-holes, the individual cube-shaped reservoirs are spaced apart (shown as “S1”) by from about 1 μm to about 5 μm.

When a plurality of through-holes are fluidly connected to a cube-shaped reservoir, the individual through-holes are spaced apart (shown as “S2”) by from about 1 μm to about 5 μm.

It should be recognized by one skilled in the art that, in the case of using the scaffold for photoreceptor cells, the processes that will become the axon of the photoreceptor cells grow downward in the through-holes. The outer segment is the specialized structure of the photoreceptor that captures light with light sensitive opsin molecules (cone and rod opsin), and these form at the opposite end (i.e., top of the capture well). The photoreceptor axons relay information from photoreceptors to send to second order retinal neurons and then ultimately to the visual centers of the brain. Growing photoreceptors in a polarized manner recapitulates their normal orientation in vivo. Transplanting photoreceptors with outer segments arranged to contact the RPE and axons poised to synapse with second order neurons will promote critical interactions between these cell types, and should lead to enhanced integration of the cells and increased functional rescue of vision.

Since a high packing density of photoreceptors is important for optimal visual acuity, the closer the cube-shaped reservoirs (and thus, the closer the cultured photoreceptor cells), the more improved the visual acuity can potentially be once the scaffold is transplanted. Accordingly, in one embodiment, the cell support layer is composed of polydimethylsiloxane (PDMS), wherein the length, L1, of the cube-shaped reservoir can be increased by stretching the cell support layer, and then allowing the cube-shaped reservoir to resume its original length upon removal of the stretching force. Particularly, with this embodiment, one or more cells are received by the cube-shaped reservoir in its stretched configuration. Once the stretching force is removed, the cells will be held within the cell reservoir. Such an embodiment is advantageous as it allows the cube-shaped reservoirs to be spaced closer together with a higher number of photoreceptor cells upon removal of the stretching force.

As discussed above, a single cube-shaped reservoir 12 is fluidly connected to a plurality of through-holes 14. As used herein, “fluidly connected to” refers to the cube-shaped reservoir in contact with a plurality of through-holes such that cells received by the cube-shaped reservoir can move/grow between the cell reservoir and the through-hole.

In another embodiment, the cell support layer is treated and/or coated such to render the polymeric layer hydrophilic. Any methods known in the art for rendering flexible polymers hydrophilic can be used without departing from the scope of the present disclosure. For example, an oxygen plasma treatment on the surface of a polymer transforms the hydrophobic surface to hydrophilic surface by introducing polar functional groups, which yields a completely wettable surface. Accordingly, methods for surface treatment include, but are not limited to, oxygen plasma treatment, ultraviolet (UV) radiation, UV/ozone treatment, corona discharges, as well as certain types of polymer or co-polymer coatings as known in the art.

The through-holes are typically included in the scaffold to allow media and/or metabolites to flow in and out of the scaffold. In suitable embodiments, the through-holes have a diameter of about 5 μm.

Methods of Preparing the Scaffold

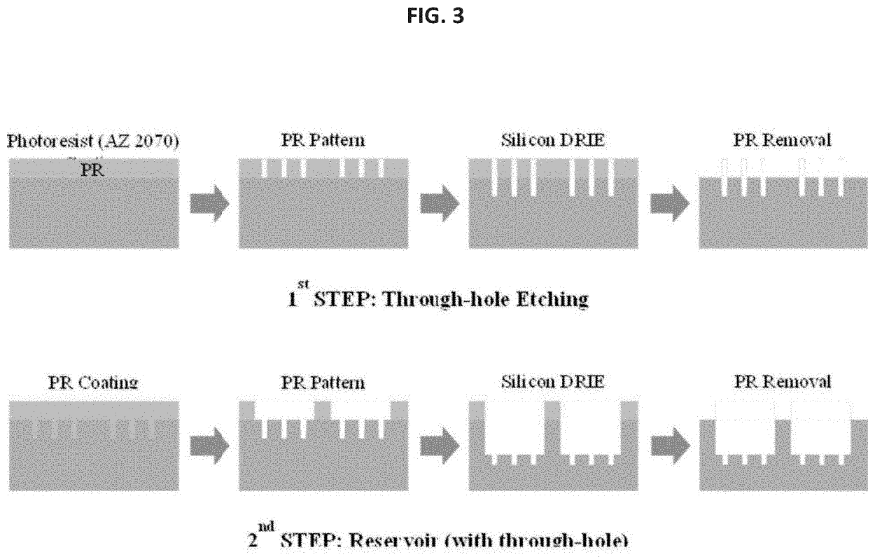

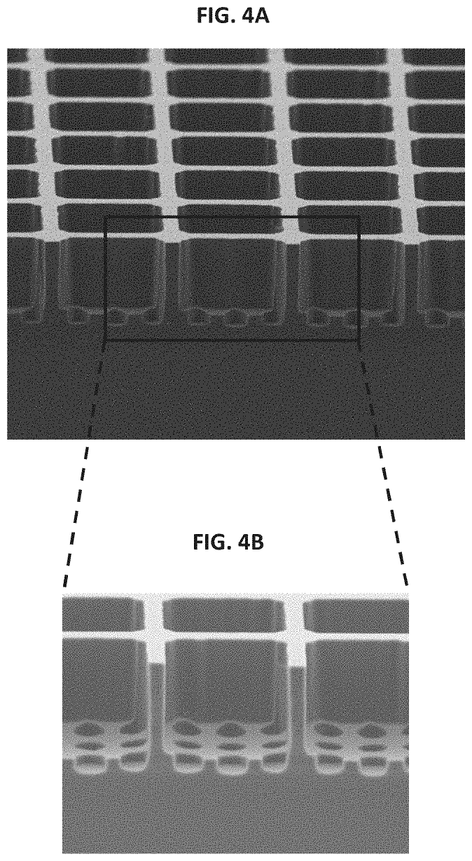

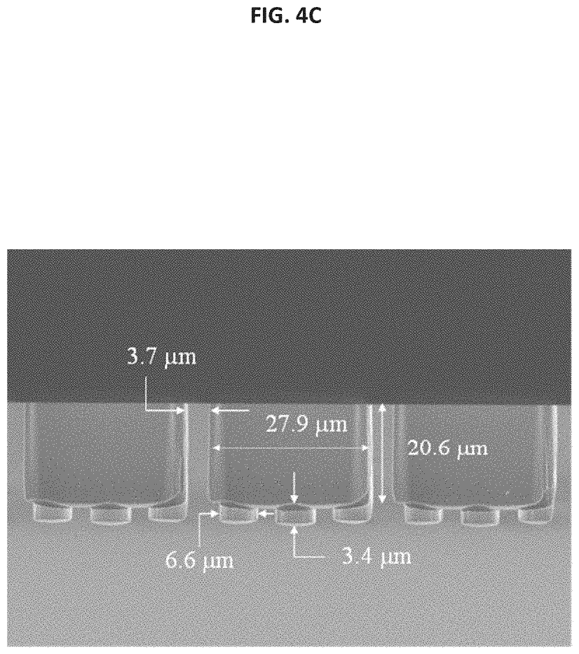

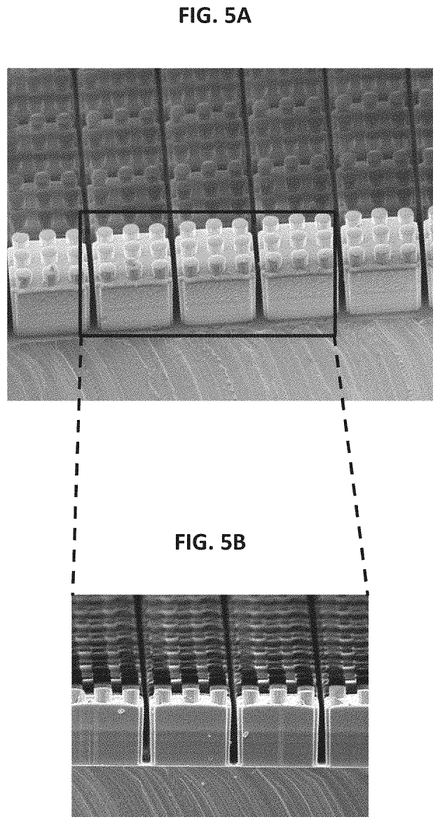

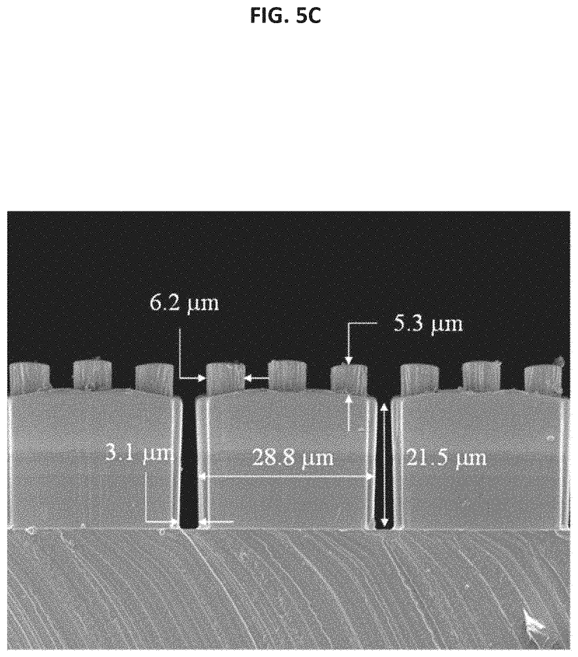

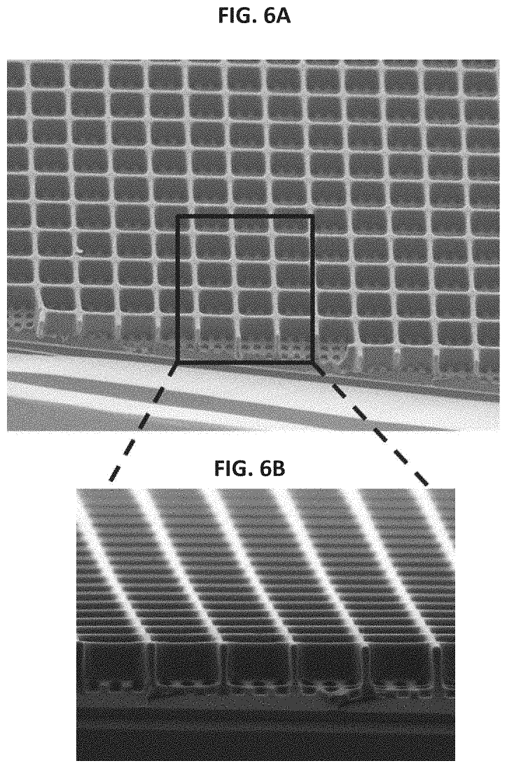

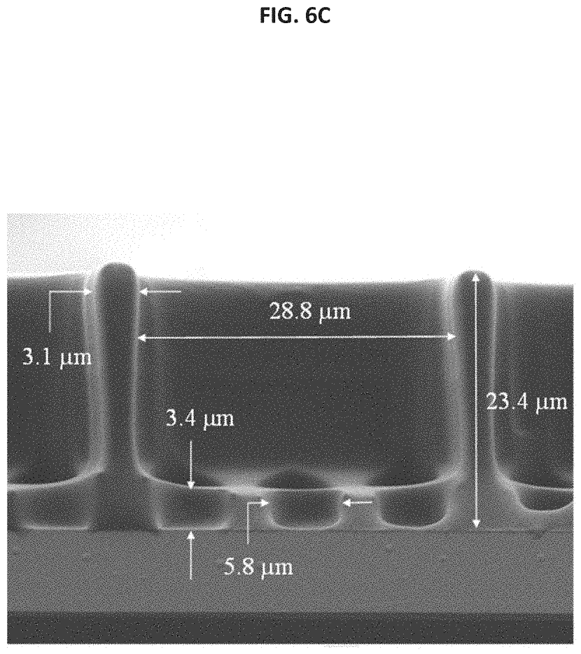

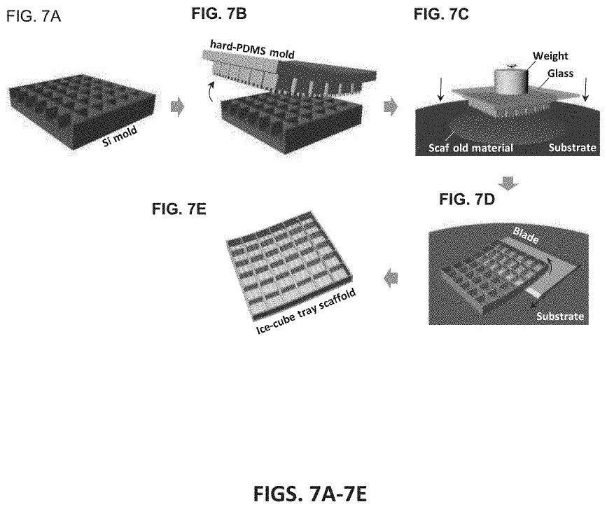

As shown in FIG. 3, the scaffolds may be prepared by forming a polymeric mold for the cell support layer. For example, to prepare the scaffold, a mold having micro-scale cubic patterns is prepared on a silicon wafer using photolithography, followed by deep reactive-ion etching (DRIE). The photoresist mask for the array of through-hole patterns is constructed first by photolithography on a silicon wafer (FIG. 3, first step). The exposed hole pattern is vertically etched via DRIE process. After the etched silicon wafer is cleaned thoroughly with solvents and plasma ashing, another photoresist mask is created on the etched silicon wafer. The second mask defines the array of cube-shaped reservoirs where the multiple through-holes are covered (FIG. 3, second step). The desired number of holes can be chosen at this step. The exposed cube area including through-holes is etched via DRIE process again. After the remaining photoresist mask on the etched silicon wafer is cleaned thoroughly with organic solvents and plasma ashing, the wafer is passivated with octafluorocyclobutane (C4F8) gas that yields a chemically inert layer similar to Teflon. The passivation layer on the etched silicon wafer prevents the casted polymer from bonding with silicon surface. Once the silicon surface becomes hydrophobic, a thick layer of hard-polydimethylsiloxane (PDMS) is poured and cured (FIGS. 5A-5C). Here, the hard-PDMS is chosen instead of soft-PDMS since it can firmly sustain the molded structures from deforming, buckling, or collapsing. Careful delamination of the hard-PDMS from the etched silicon wafer generates a robust master mold for creating scaffolds (FIG. 7B). Subsequently, the hard-PDMS master mold is further treated with self-assembled monolayer (SAM) of silane via vacuum evaporation at room temperature. Thereafter, a drop of soft-PDMS is casted on top of the mold, which is then pressed upside down on a handling substrate such as glass, silicon wafer, etc. (see FIG. 7C), followed by a curing process in an oven (FIG. 6A, FIG. 6B, and FIG. 6C). Once the soft-PDMS is fully cured, the hard-PDMS master mold is carefully delaminated from the handling substrate and the scaffold generated on the substrate is picked up and mounted on a transwell for cell growth. More particularly, the outer edge of the scaffold was attached to the transwell insert using a soft PDMS as a glue. The cell seeding area of the transwell insert was 78.5 mm² (internal diameter: 5 mm). It should be understood the size of the transwell insert can vary without departing from the present disclosure.

While described herein as preparing the scaffolds using photolithography, DRIE and molding, it should be understood that any means for preparing polymeric scaffolds as known in the art can be used without departing from the present disclosure. Other suitable methods include, for example, direct printing using a 3D printer or molding using a micro-injection molding machine.

Further, while described herein as using PDMS, it should be understood that other biocompatible or biodegradable polymers, such as, for example, synthetic rubbers such as silicone rubbers, polyurethane rubber, styrene butadiene rubber, acrylonitrile butadiene rubber, natural rubbers (e.g., poly-cis-isoprene), epoxies (e.g., SU-8), polyimides, poly(p-xylylene) (e.g., Parylene), polyesters (e.g., poly(lactic-co-glycolic acid) (PLGA), polylactic acid (PLA), polyglycolic acid (PGA), polycaprolactone (PCL), poly(lactide-co-ε-caprolactone (PLCL), poly(glycerol sebacate) (PGS), poly(glycerol-citrate) (PGC), poly(glycerol-sebacate-citrate) (PGSC)), thermoplastic elastomers (e.g., thermoplastic polyurethane, thermoplastic copolyester, thermoplastic polyamide), aliphatic polycarbonate, polyurethanes, polysaccharides (e.g., chitosan), and combinations thereof, can be used in addition to or as an alternative to PDMS without departing from the scope of the present disclosure.

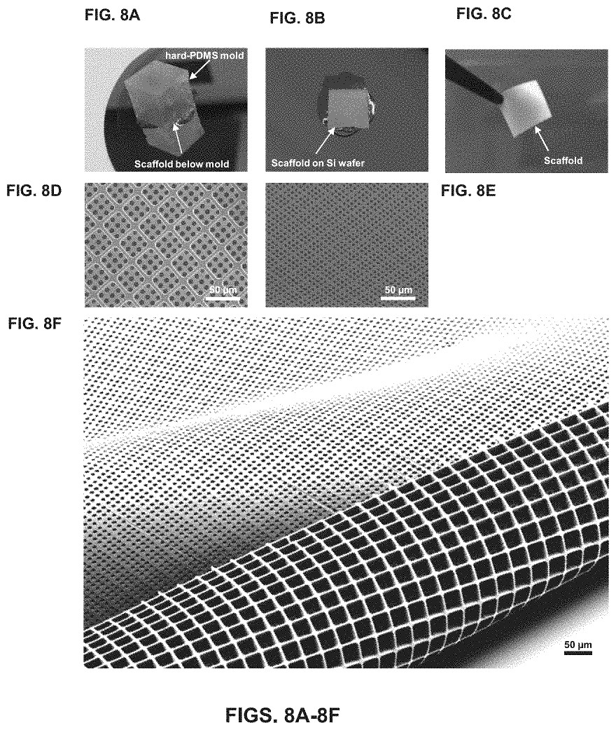

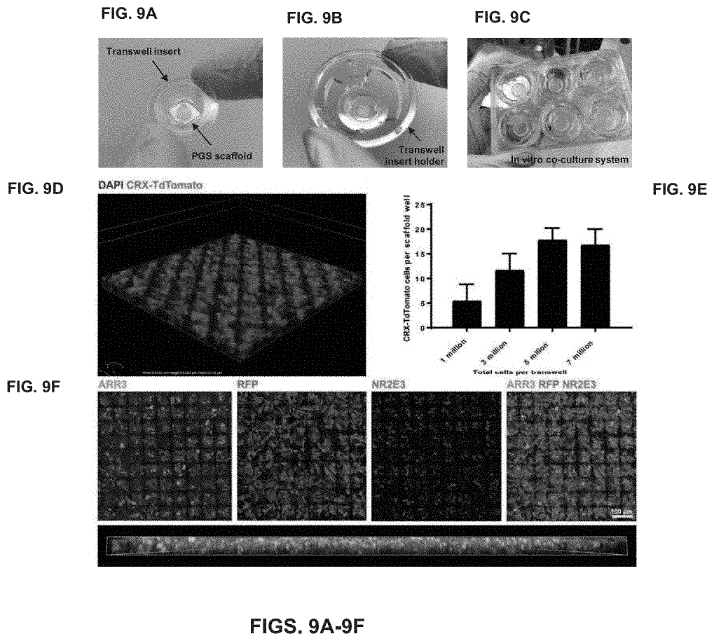

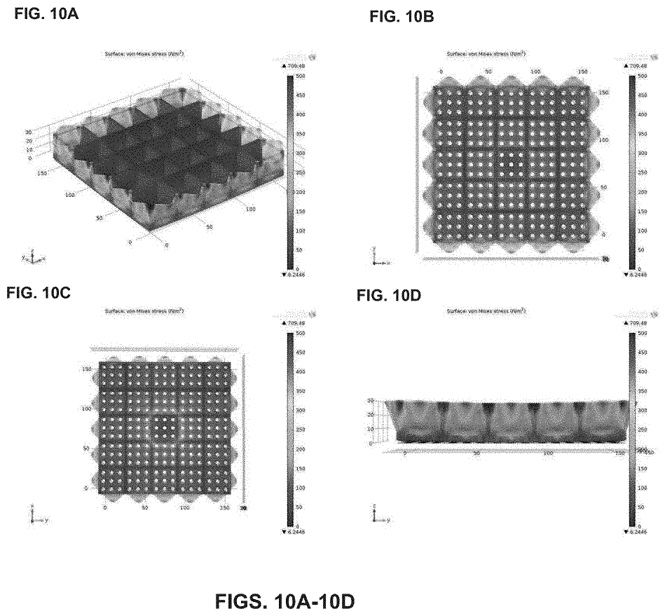

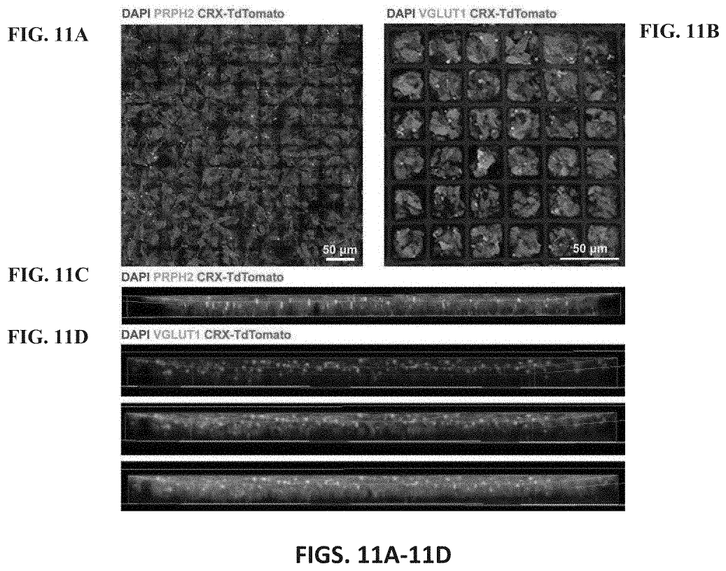

For example, in FIGS. 8A-8F a scaffold is prepared using poly(glycerol sebacate) (PGS).

Uses of the Scaffold System in Cell Culture

Advantageously, the scaffolds of the present disclosure can be used for cell culturing, transplantation, developmental modeling, disease modeling, and for drug screening.

When used for cell culture, the cell culture scaffold system generally includes a scaffold including a cell support layer. The cell support layer is typically comprised of a biocompatible or biodegradable polymer, and in some particularly suitable embodiments, the polymer is biodegradable. The polymer may be porous or nonporous. Suitable polymers include, but are not limited to, synthetic rubbers such as silicone rubbers, polyurethane rubber, styrene butadiene rubber, acrylonitrile butadiene rubber, natural rubbers (e.g., poly-cis-isoprene), epoxies (e.g., SU-8), polyimides, poly(p-xylylene) (e.g., Parylene), polyesters (e.g., poly(lactic-co-glycolic acid) (PLGA), polylactic acid (PLA), polyglycolic acid (PGA), polycaprolactone (PCL), poly(lactide-co-ε-caprolactone (PLCL), poly(glycerol sebacate) (PGS), poly(glycerol-citrate) (PGC), poly(glycerol- sebacate-citrate) (PGSC)), thermoplastic elastomers (e.g., thermoplastic polyurethane, thermoplastic copolyester, thermoplastic polyamide), aliphatic polycarbonate, polyurethanes, polysaccharides (e.g., chitosan), and combinations thereof. In one particular aspect, the polymer is PDMS. In another particular aspect, the polymer is PGS

The cell support layer includes at least one cube-shaped reservoir fluidly connected to a plurality of through-holes, the at least one cube-shaped reservoir fluidly connected to the through-holes extends through the cell support layer. The cube-shaped reservoir is as described above. Particularly, the cube-shaped reservoir has a length that is larger than the diameter of the through-hole.

As noted above, it should be understood by one skilled in the art that the cell support layer can include more than a single cube-shaped reservoir connected to the plurality of through-holes. Particularly, the cell support layer of the cell culture scaffold may include a plurality of cube-shaped reservoirs, each separately fluidly connected to a plurality of through-holes without departing from the scope of the present disclosure.

When a plurality of cube-shaped reservoirs are connected to through-holes, the individual cube-shaped reservoirs are spaced apart by from about 1 μm to about 5 μm, including from about 2 μm to about 3 μm.

Typically, the cell support layer has a thickness ranging from about 20 μm to about 45 μm, including from about 20 μm to about 35 μm, and including about 23 μm.

In some embodiments, the cell support layer is treated and/or coated such to render the polymer hydrophilic as described above.

The cell culture scaffold system further includes at least one cell in the cell support layer. Cells received by the cube-shaped reservoir of the cell support layer can move/grow between the cube-shaped reservoir and the through-hole of the cell support layer. Any cells as known in the art for use in a scaffold system for in vitro developmental and disease studies, as well as for drug screening, could be used with the cell culture scaffold system of the present disclosure. Particularly, suitable cells include photoreceptor cells, retinal pigment epithelium (RPE) cells, bipolar cells, ganglion cells, and combinations thereof.

Use of the cell culture scaffold system results in the formation of an organized multi-cellular construct that mimics the cellular structure and organization observed in vivo, allowing for improved grafted cell survival, integration, and functional visual rescue. Further, these structures prevent reflux similar to those currently found with the use of bolus injections.

More particularly, in one suitable embodiment, the scaffold system includes at least one photoreceptor cell and at least one retinal pigment epithelium (RPE) cell in the cell support layer. Photoreceptors are the gatekeepers of vision, and they capture and transduce photos into electro-chemical signals to be processed by the retina and visual centers of the brain. Photoreceptors are highly polarized, specialized cell types with apical outer segments containing light sensing photo-pigments and basal axon terminals. As naturally found, adjacent to the photoreceptors are the RPE cells, supportive cells required for photoreceptor health and function. Particularly, an RPE monolayer provides cellular and structural cues for photoreceptor polarization. Particularly, the cell support layer is sized such to contain a monolayer of RPE at the bottom of each cube-shaped reservoir and a layer of photoreceptor cells on top of the RPE. For this combined photoreceptor +RPE scaffold, the underlying through-holes will support RPE function such as metabolite, water, and waste transport. In this embodiment, once placed, the photoreceptors can start to grow their processes, which will ultimately become their outer segments towards the RPE. These polarized photoreceptors are then poised for in vitro testing or transplantation.

For a photoreceptor only application, the through-holes at the bottom of the scaffold will promote polarization, by directing extension of photoreceptor axons.

When used for transplantation, the cell culture scaffold system as described above is used to culture the cells to form a construct and then the cultured construct is transplanted to the eye of a subject using standard vitreoretinal surgical techniques. Suitable diseases and/or conditions that these constructs could be potentially used for include all inherited or acquired diseases or retinal injuries involving dysfunction and/or death of photoreceptors or the dysfunction and/or death of the RPE and photoreceptors. These include, but are not limited to, age-related macular degeneration (“dry” or “wet” AMD), retinitis pigmentosa, retinal detachment, cone dystrophies, cone-rod dystrophies, Usher's syndrome, Best's disease, choroideremia, gyrate atrophy, myopic degeneration, sorsby's fundus dystrophy, doyne honeycomb macular dystrophy, and Stargardt macular dystrophy.

When the scaffold system is used for drug screening, candidate agents are added to a culture media. Cell health and survival can then be assessed using standard techniques. Additionally, the scaffold system makes it possible to examine the effects of candidate agents on cellular structures that do not typically develop without the aid of this scaffold system, such as the development of photoreceptor outer segments. Many blinding retinal disorders originate in the photoreceptor outer segment, involving an absence of photoreceptor specific protein expression, misfolded proteins, incorrect packaging of proteins, or ectopic localization of these proteins within the photoreceptor. These components of disease and potential candidates are difficult to assess in a typical two-dimensional culture system (cells grown on a flat surface). Furthermore, many diseases require modeling of RPE-photoreceptor interactions. Investigating these interactions is possible with this system, but not with traditional two-dimensional culture, as RPE and photoreceptors will not grow on top of each other in a two dimensional culture.

Various functions and advantages of these and other embodiments of the present disclosure will be more fully understood from the examples shown below. The examples are intended to illustrate the benefits of the present disclosure, but do not exemplify the full scope of the disclosure.

### EXAMPLE 1

In this Example, to demonstrate the mechanical stability of the PGS scaffold made using the processes of the present disclosure, the von Mises stress distribution in the ice-cube tray PGS scaffold was numerically solved using a finite element simulation software (Comsol Multiphysics 4.2, Comsol Ltd).

The applied tensile force in the x- and y-directions were Fₓ=±5 N/m² and F_{y}=±5 N/m², respectively. The material parameters used for PGS polymer were as follows: E=1.3 Mpa, v=1060 kg/m3, p=0.49. Here, E is the Young's modulus, v is the Poisson's ratio, and p is the density.

### EXAMPLE 2

In this Example, micro-patterned “ice-cube tray” scaffolds are analyzed to determine if the scaffolds can act synergistically with existing host retinal structures, supporting prearranged orientation of seeded photoreceptors (PRs).

Laminin-coated scaffolds seeded with hPSC-PRs were fixed in 4% paraformaldehyde and processed for immunohistochemistry to assess expression of rod, cone, outer segment, and synaptic protein markers. Confocal z-stacks of immunostained scaffolds were captured at 20× magnification with a Nikon AlR confocal microscope. 3D z-stack reconstruction, maximum intensity projection image generation, and cell capture quantification were performed with Nikon Elements software. Statistical analyses were performed with GraphPad Prism.

This unique feature of the “ice-cube tray” design ensures that VGLUT1 is maximally expressed in the top portion of the scaffold, which will face the host inner nuclear layer of interneurons and is designed to more easily facilitate synaptic connections in the host retina. This orientation also ensures that the base of the scaffold lies directly above the host retinal pigment epithelium, which is the primary cell type that will be responsible for scaffold degradation in the subretinal space. 3D renderings (FIGS. 12C & 12D) are 644 μm×644 μm×20 μm.

What is claimed is:

1. A scaffold comprising a cell support layer comprising at least one cube-shaped reservoir, wherein the at least one cube-shaped reservoir is fluidly connected to a plurality of through-holes.

2. The scaffold of claim 1, wherein the cell support layer comprises a biocompatible flexible polymer.

3. The scaffold of claim 2, wherein the biocompatible flexible polymer is selected from the group consisting of silicone rubber, polyurethane rubber, styrene butadiene rubber, and acrylonitrile butadiene rubber, natural rubber, thermoplastic elastomer, epoxy, polyimide, polyurethane, polyamide, polyester, polysaccharide, parylene, and combinations thereof.

4. The scaffold of claim 2, wherein the biocompatible flexible polymer is polydimethylsiloxane (PDMS).

5. The scaffold of claim 1, wherein the cell support layer comprises a biodegradable flexible polymer.

6. The scaffold of claim 1, wherein the at least one cube-shaped reservoir has a length ranging from about 15 μm to about 100 μm.

7. The scaffold of claim 1, wherein the at least one cube-shaped reservoir has a height ranging from about 5 μm to about 100 μm.

8. The scaffold of claim 1, wherein a single through-hole of the plurality of through-holes has a diameter of from about 1 μm to about 7 μm.

9. The scaffold of claim 1, wherein a single through-hole of the plurality of through-holes has a height of from about 1 μm to about 25 μm.

10. The scaffold of claim 1, wherein the cell support layer comprises at least a first cube-shaped reservoir and a second cube-shaped reservoir, the first cube-shaped reservoir spaced from about 1 μm to about 5 μm apart from the second cube-shaped reservoir.

11. A cell culture scaffold system comprising a scaffold comprising a cell support layer comprising at least one cube-shaped reservoir, wherein the at least one cube-shaped reservoir is fluidly connected to a plurality of through-holes, and at least one cell in the at least one cube-shaped reservoir.

12. The cell culture scaffold system of claim 11, wherein the cell in the at least one cube-shaped reservoir is a photoreceptor cell.

13. The cell culture scaffold system of claim 12, wherein the at least one cube-shaped reservoir comprises a single photoreceptor cell.

14. The cell culture scaffold system of claim 12, wherein the at least one cube-shaped reservoir comprises at least two photoreceptor cells.

15. The cell culture scaffold system of claim 11, wherein at least one cube-shaped reservoir comprises a monolayer of retinal pigment epithelial cells and a layer of photoreceptor cells on top of the monolayer of retinal pigment epithelial cells.
